(19) United States
(12) Patent Application Publication
(10) Pub. No.: US 2005/0058704 A1
(43) Pub. Date: Mar. 17, 2005

(54) Capsule containing active substance pellets

(75) Inventors: Roland Schneider, Sprendlingen (DE); Sergej Anschuetz, Ruemmelsheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/932,839

(22) Filed: Sep. 2, 2004

(30) Foreign Application Priority Data
Sep. 3, 2003 (EP) 03019577

(51) Int. Cl. A61K 9/54; A61K 9/46

(52) U.S. Cl. 424/458

(57) ABSTRACT

The invention relates to a capsule containing different active substance pellets with at least two different active substances which differ in their release profile in the gastro-intestinal tract, these active substances being selected from among the vitamins, minerals, trace elements, unsaturated fatty acids, amino acids and/or plant extracts and substances. The different release profiles represent rapid, moderate and/or slow dissolving of the active substance pellets. This controlled release leads to a targeted absorption of the active substances in the different absorption areas of the gastro-intestinal tract. Using the preparation provided according to the invention it is also possible to improve bioavailability to a high degree even when a large number of active substances are present.

The invention relates to capsules containing pellets of active substance comprising at least three different active substances, the active substances being selected from among the nutrients such as e.g. vitamins, minerals, trace elements, unsaturated fatty acids and amino acids or the plant substances and extracts.

### BACKGROUND TO THE INVENTION

It is a well known fact that active substances should be released where they can most effectively be absorbed by the body. However, this is not so easily put into practice.

Formulations are known which are coated so as to release the drugs which they contain over a longer time or after a delay, thereby producing a so-called sustained or delayed release effect. These pharmaceutical compositions must be distinguished from those with a controlled or modified release of active substance. These latter forms are used particularly when the active substances specifically have to reach a remote absorption site. The supply of active substance is determined not only by the amount administered but also by the bioavailability. For each active substance there is an optimum release or absorption site. It is to be assumed that as a result of different pH values, temperatures etc. during the transit through the gastro-intestinal tract, losses will occur, which can be prevented by means of the present invention. This is a problem particularly when administering different active substances with different absorption sites.

In the case of a preparation having a number of active substance ingredients there is also the danger that the active substances present will affect one another, as a result of which their activity will be inhibited or even blocked and consequently their spectrum of activity is lowered or their potential is lost. Food ingredients may also reduce the absorption of certain active substances. For example the oxalic acid in spinach or the phytic acid in wholegrain products may reduce the absorption of calcium. On the other hand, absorption may also be increased. Thus the simultaneous administration of vitamin D increases the absorption of calcium in the small intestine. To utilise the active substances to the full it would therefore be necessary to have a complex and precisely timed sequence for taking the various individual substances, which is out of the question for daily use because of the complexity and the amount of time involved.

Therefore the prior art contains some suggestions as to how to master the problems mentioned above:

WO 01/72286 A1 provides a therapeutic formulation in the form of pearls for oral administration in which the pharmaceutical composition is released at controlled rates. The pearls are made up of three layers, a spherically extruded inner core which contains the medicament to be released after a delay, an outer layer which contains a medicament to be released immediately, and an intermediate layer between the two layers, which can additionally control the release of the medicament in the inner core. Accordingly, a formulation is proposed in which several different rates of release are provided in each individual pellet.

According to the teaching of U.S. Pat. No. 3,939,259 therapeutic preparations are described consisting of particles with degradable coatings of zein and shellac which contain a therapeutically active material, the release being controlled by varying the layer thickness. Vitamins may also be used.

International Patent Application WO 98/19667 proposes a “sustained release” formulation for the oncologically-active drug DFMO.

International Patent Application WO 00/69420 describes a preparation for vitamin C in which the vitamin C is released in the form of microcapsules in the gastro-intestinal tract in 3 release pulses.

Moreover, German Utility Model DE 202 02 984 U1 relates to a multi-chamber capsule for the time-delayed release of elementary nutrients in the gastro-intestinal tract, the capsule consisting of several chambers arranged inside one another or joined to one another. The individual chambers contain different active substances, such as vitamins, trace elements, minerals and amino acids. Thus, capsules are obtained which are nested inside one another in a complicated manner. This technical solution appears to be technically highly complicated and furthermore difficult to manufacture on account of the complex structural elements and not economically viable for industrial use.

European Patent Application EP 0 820 703 describes formulations in which lipophilic active substances are released quickly (in less than an hour) and hydrophilic substances are released slowly (over eight hours). There is no mention of the particular release site in this European Patent Application.

The provision of a system which allows the formulation and controlled release, in terms of the release site, of a larger number of different active substances, without having to resort to the complicated structures mentioned above, is therefore still an unsolved problem in the prior art.

The present invention is thus based on the problem of providing a simplified formulation which yields an improved bioavailability for active substances, particularly for nutrients such as vitamins, minerals, trace elements, unsaturated fatty acids, amino acids or plant extracts and substances. The preparation should offer flexibility in terms of the release profiles, so that both rapid and delayed release is possible at the absorption sites in the stomach and in the corresponding sections of the intestines, in controlled and targeted manner. The preparation should also be easy and economical to produce.

### DETAILED DESCRIPTION OF THE INVENTION

The problem mentioned above is solved according to the invention by a capsule containing active substance pellets with at least three different active substances which differ in their release profile in the gastro-intestinal tract, the active substance or substances being selected from among the vitamins, minerals, trace elements, unsaturated fatty acids, amino acids, plant active substances and plant extracts. In contrast to the disclosure of WO 01/72286, in which a number of different rates of release are achieved in each individual pellet, a particular active substance pellet according to the invention has only one release profile. The release profile may constitute fast, moderate and/or slow dissolving of the pellets of active substance. Thus, capsules are provided which contain a number of different pellets, each having a different release profile. Consequently, the active substances contained therein are released in controlled manner in the gastro-intestinal tract at the point where they are most effectively absorbed.

The invention thus relates to a capsule containing pellets of active substance which differ in their release profile in the gastrointestinal tract, these pellets containing at least two different active substances which are selected from among the vitamins, minerals, trace elements, unsaturated fatty acids, amino acids and/or plant extracts and substances, while at least three groups of pellets each having the same release profile are present and the release of the particular active substances takes place over the entire absorption area in the gastro-intestinal tract (group I), only in the duodenum or only in the duodenum and in the jejunum (group II), or only in the jejunum, only in the jejunum and ileum or only in the ileum (group III).

The different release profiles may by adjusted by means of the composition and/or structure of the pellets of active substance. Preferably, particular release profiles are achieved using different coatings for the pellets, for example by varying the thickness of the coating and/or by the choice of composition of the coating, in order to utilise a pH-controlled release, for example. Typical coatings commonly used in pharmacology and technology are film and sugar coatings. Coatings with delayed release (delayed-release tablets) are for example diffusion coatings or soluble coatings such as a coating soluble in the gut, i.e. a coating which is substantially resistant to the gastric juices but dissolves during its passage through the gut. Examples of suitable coatings include shellac, zein, gelatine, cellulose acetate phthalate, hydroxypropylmethylcellulose phthalate, ethyl cellulose, stearic acid, carnauba wax, glycerol behenate, polymers of acrylic acid esters and methacrylic acid esters, such as Eudragit® coating materials, etc.

In individual cases it may be advantageous if no coating of zein and shellac is used. Coating technology in galenic pharmacology falls within the ambit of the skilled man and consequently no further details are required.

The present invention therefore makes it possible to select the active substances contained in the pellets in accordance with the delayed and site-controlled releases. For example, a number of pellets may have the same release profile, so that one or more groups of pellets are present which have a fast, moderate and/or slow release profile. There is also the possibility of releasing active substances through the entire gastro-intestinal tract or the entire small intestine, i.e. there may be an immediate, rapidly occurring and also a time-delayed release. The above variants of fast, moderate and/or slow release are therefore to be understood not only as alternatives but also in conjunction with one another, as it is possible and desirable to have a number of release profiles when using several active substances.

The term “pellet” according to the present invention is intended to denote every possible preparation for oral administration, such as extruded materials, tablets, particles, pearls, granules, coated tablets and the like, which because of their size can be administered in capsules without causing unwanted problems in swallowing on account of the size of the capsule containing them. Preferably, the pellets are extruded active substances and excipients.

The capsule according to the invention serves only as a neutral carrier medium which is dissolved as necessary at the desired release site in the gastro-intestinal tract, in order to release the pellets of active substance contained therein and allow them their corresponding release profiles. Both hard and soft capsules may be used, the latter being preferred.

Preferably the capsules according to the invention contain at least one vitamin, particularly at least 2 vitamins, most preferably 2 to 10 vitamins.

To illustrate the present invention the active substances are selected from among the vitamins and minerals, by way of example. Other possible ingredients come from the trace elements, the unsaturated fatty acids, the amino acids and/or the plant substances and extracts. Of the vitamins, both water-soluble and fat-soluble vitamins may be used. Suitable vitamins are water-soluble vitamins, such as vitamin C, e.g. L-(+)-ascorbic acid, calcium ascorbate, potassium ascorbate, 6-palmitoyl-L-ascorbic acid; vitamin B1, e.g. thiamine hydrochloride, thiamine mononitrate; vitamin B2, e.g. riboflavin, riboflavin-5′-phosphate sodium; vitamin B6, e.g. pyridoxine hydrochloride, vitamin B 12, e.g. cyanocobalamine; vitamin H, e.g. D-biotin; folic acid; vitamin PP (niacin), e.g. nicotinamide, nicotinic acid; pro-vitamin B5, e.g. panthenol (D and DL forms), ethylpanthenol and calcium-D-pantothenate. Suitable fat-soluble vitamins are e.g. vitamin A, such as vitamin A-palmitate, vitamin A-acetate, vitamin A-propinate, trans-retinol; vitamin D, e.g. ergocalciferol, cholecalciferol, cholecalciferol-cholesterol; vitamin E, e.g. alpha-tocopherol, alpha-tocopherylacetate, alpha-tocopherylic acid succinate (D and DL forms); vitamin K, such as vitamin K1, e.g. phytomenadione, and carotenoids (provitamin), e.g. lycopene, zeaxanthine, lutein, alpha-carotene, beta-carotene, apocarotinal, gamma-carotene and beta-cryptoxanthine. Riboflavin is preferably used in salt form, e.g. as riboflavin phosphate, as the salt form has greater stability.

Preferably, the capsule according to the invention contains 20 to 150, particularly 40 to 120, most preferably about 100 mg of vitamin C, 0.5 to 2.0, particularly 0.8 to 1.5, most preferably about 1.1 mg of vitamin B1; 0.8 to 2.5, particularly 1.0 to 2.0, most preferably about 1.2 mg of vitamin B2, 0.8 to 2.5, particularly 1.0 to 2.0, most preferably about 1.5 mg of vitamin B6, 0.8 to 5.0, particularly 1.2 to 3.5, most preferably about 3.0 μg of vitamin B12, 5 to 200 particularly 10 to 100, most preferably about 50 μg of D-biotin; 50 to 600, particularly 100 to 500, most preferably about 400 μg of folic acid; 5.0 to 50.0, particularly 10 to 30, most preferably about 16 mg of vitamin PP (niacin); 1.0 to 50.0, particularly 2 to 20, most preferably about 6 mg of panthenol, 300 to 1300, particularly 500 to 1000, most preferably about 800 μg of vitamin A, 0 to 20, particularly 2.5 to 15.0, most preferably about 5 μg of vitamin D, 0.1 to 300.0, particularly 5.0 to 15.0, most preferably about 12 mg of vitamin E, and 10 to 200, particularly 25 to 150, most preferably about 75 μg of vitamin K.

According to a particularly preferred embodiment of the invention the active substances or nutrients may be divided into three groups, i.e. the active substances which are released over the entire absorption area of the gastro-intestinal tract (group I), the active substances which are released in the duodenum or in the duodenum and in the jejunum (group II) and those which are released in the jejunum, in the jejunum and ileum or in the ileum (group III). According to this classification, which represents the optimum absorption site for a selected active substance, the particular active substances is advantageously assigned to those pellets which have the appropriate release profile in order to deliver the active substances to the desired absorption site.

This principle will be illustrated using vitamins as an example. Vitamins of group I are selected for example from niacin, pantothenic acid, vitamin D and biotin, vitamins of group II are selected from vitamin A, riboflavin, thiamine and folic acid and vitamins of group III are selected from vitamin B6, vitamin C and vitamin K. Obviously, this is not an exhaustive list but serves purely as an illustration. Other examples will be familiar to those skilled in the art.

According to this variant of the invention each active substance pellet contains either one or more vitamins of group I, II or III. However, combinations may also be prepared. As the vitamins of group I are intended to be released and absorbed over the entire range, there is also the possibility that two types of active substance pellets are present, one type containing vitamins of group I and II and the other type containing vitamins of group I and III. Thus, the vitamins that are to be released throughout the range are located both in the pellets with the more direct, i.e. rapid release profile and in those with the more indirect and/or slow release profile.

“Rapid” release means that the desired plasma concentration of the vitamins is achieved within a relatively short time, for example about 0.5 to 1 hour after administration or, in the case of the active substances that are absorbed in the small intestine, after administration and passing through the stomach, with substantially the entire vitamin content of a pellet being released all at once. The release and absorption take place predominantly in the stomach or in the upper part of the small intestine, while the quantity and nature of food eaten beforehand and the time frame of when the last meal was eaten are also of importance in this respect. The terms “fast”, “moderate” and “slow” release are hence relative terms and serve as parameters without representing any absolute values.

The assignment of the active substances, such as for example the vitamins, to the desired release profiles determines the absorption site at which the release then takes place, which means that great care has to be exercised here. The wrong release site obviously reduces the activity potential of the active substance considerably.

The invention also relates to a process for preparing the capsules according to the invention, comprising
selecting the active substances in the form of e.g. vitamins, minerals, trace elements, unsaturated fatty acids, amino acids or plant substances or extracts on the basis of their respective absorption sites;
producing several groups of active substance pellets with different release profiles for the respective absorption sites, incorporating the vitamins and optionally other active substances which are divided between the groups of active substance pellets according to their release profiles and absorption sites, and
introducing the active substance pellets into a capsule.

The active substance(s) in the form of one or more active substances are preferably contained in the active substance pellets of the invention in an amount of about 0.001 to about 99% by weight.

The active substance pellets contained in the capsules according to the invention may contain other active substances in addition to the vitamins mentioned above, such as minerals, trace elements, plant extracts, amino acids and unsaturated fatty acids.

Examples of minerals include calcium, such as calcium hydrogen phosphate, calcium citrate; magnesium, such as magnesium carbonate, magnesium lactate; potassium, such as potassium chloride, potassium sulphate; phosphorus, such as calcium phosphate; and chloride, such as potassium chloride, magnesium chloride. Suitable trace elements may be both inorganic and organic salts. Examples include: iron, such as iron fumarate, iron citrate, iron lactate; zinc, such as zinc lactate, zinc citrate, zinc oxide; iodine, such as sodium iodate, potassium iodide; copper, such as copper gluconate, copper sulphate; manganese, such as manganese citrate, manganese sulphate; molybdenum, such as sodium molybdate, ammonium molybdate; selenium, such as sodium selenate, sodium selenite; chromium, such as chromium chloride, chromium citrate; silicate, such as silicon dioxide, sodium silicate; and fluoride, such as sodium fluoride. According to the invention calcium, magnesium, iron, zinc, copper, manganese, selenium, phosphorus and iodine, the salts thereof and mixtures thereof are particularly preferred as the minerals and trace elements.

Preferably, the capsule according to the invention contains one or more mineral pellets containing one or more of the following minerals in the amounts specified:

100 to 2500, particularly 500 to 1200, most preferably about 1000 mg of calcium, 50 to 1000, particularly 100 to 600, most preferably about 375 mg of magnesium, 500 to 5000, particularly 1500 to 3000, most preferably about 2000 mg of potassium, 100 to 2000, particularly 300 to 1500, most preferably about 700 mg of phosphorus, 500 to 7500, particularly 750 to 5000, most preferably about 800 mg of chloride, 0.5 to 50, particularly 1.0 to 20, most preferably about 2.0 mg of manganese, 5.0 to 50, particularly 7.5 to 25, most preferably about 5.0 mg of iron; 1.0 to 50, particularly 2.5 to 20, most preferably about 5.0 mg of zinc; 10 to 500, particularly 100 to 200, most preferably about 150 μg iodine, 0.1 to 5.0, particularly 0.5 to 5.0, most preferably about 1.0 mg of copper, 0.5 to 15.0, particularly 1.0 to 12.5, most preferably about 2.0 mg of manganese, 10 to 500, particularly 20 to 450, most preferably about 50 μg of molybdenum, 5 to 250, particularly 10 to 200, most preferably about 55 μg of selenium, 5 to 200, particularly 10 to 150, most preferably about 40 μg of chromium, and 0.5 to 15.0, particularly 1.0 to 10.0, most preferably about 3.5 mg of fluoride.

Plant extracts which may be used are preferably selected from Ginkgo biloba, Panax Ginseng, Vitis vinifera, i.e. vine leaves and also grape seeds, Guarana, Cimicifuga racemosa and Turnera aphrodisiaca, i.e. damiana leaves or special plant extracts which are rich in kaempferol or kaempferol glucosides and/or are rich in lutein or other flavonoids. Depending on the desired use amino acids may also be added, of which lysine, arginine and taurine are particularly preferred. Unsaturated fatty acids may also be present, such as ω-fatty acids, particularly DHA.

The active substance pellets contained in the capsules according to the invention are composed such that the normal single dose of a nutrient, for example divided between a number of active substance pellets or contained in one active substance pellet, is in the range from about 10 to 300%, preferably about 100% of the recommended daily dose. The additional amount of minerals and trace elements present is preferably in the range from about 1 to 100% of the recommended daily dose.

The active substance pellets of the present invention may contain other excipients, such as plasticisers, e.g. glycerol, polyethyleneglycol, castor oil and acetylated monoglycerides; pH modifiers, such as potassium hydrogen phosphate; fillers, such as calcium carbonate; binders, such as microcrystalline cellulose, stabilisers, lubricants, disintegrants, breakdown agents, colourings and the like.

The advantages associated with the present invention are numerous:

There is no need to administer numerous tablets with different release characteristics. This is all the more important with multi-component formulations in which a variety of active substances are used. For example, a multivitamin preparation administered as a food supplement may contain 10 vitamins, 5 minerals and 12 trace elements which are intended for rapid release and an equal number of active substances which are intended to be released more slowly. This comprises 54 different active substances with different release characteristics, which would totally overwhelm the consumer if he had to take them all individually.

Therefore, taking the multicomponent capsule according to the invention, containing different vitamins and other active substances or nutrients, is very convenient and comfortable for the consumer or patient and the daily dose is reduced to one capsule or a few capsules. Moreover the active substances are actually absorbed virtually without any effect on one another. The active substances contained therein, particularly the vitamins and other nutrients as well as plant extracts and substances, are released in the gastro-intestinal tract at the point where they are absorbed most effectively, i.e. their bioavailability is significantly improved. As a result of the controlled release of the different active substances the dosage can be adjusted much more accurately, thereby reducing, i.e. optimising, the amount of active substance, and preventing overdoses. Thanks to the controlled release according to the invention each active substance can be delivered to the desired absorption site, where it can develop its optimum spectrum of activity.

Moreover, active substances which are usually incompatible with one another can also be administered together, as they are kept separate from one another. For example, some trace elements and minerals are incompatible with some fat-soluble vitamins. According to the invention these may be administered together.

The present invention therefore provides a novel preparation which yields optimum bioavailability for each individual active substance, even when there is a large number of active substances.

### DESCRIPTION OF THE FIGURES

The accompanying Figures illustrate the teaching according to the invention without restricting it thereto. Specifically, they show:

FIG. 1: a diagrammatic representation of a preparation from the prior art according to WO 01/72286;

FIG. 2: a diagrammatic representation of an inventive embodiment of a capsule according to the invention,

FIG. 3: the absorption of selected vitamins in different parts of the small intestine

FIG. 4: four embodiments of capsules from the prior art as well as two embodiments of a capsule according to the invention, and

FIG. 5: three different embodiments of pellets for a capsule according to the invention.

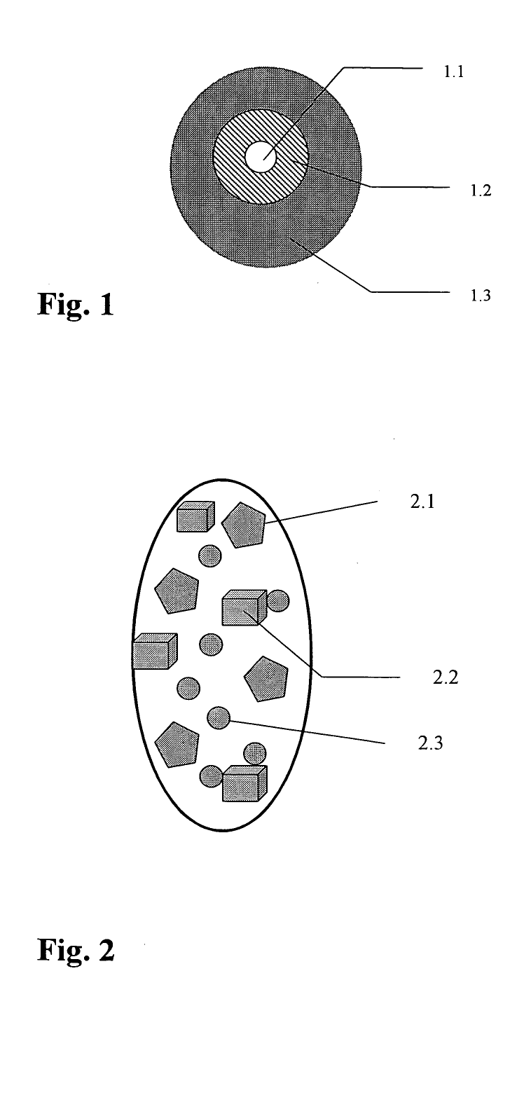

FIG. 1 diagrammatically shows a preparation according to WO 01/72286 in section, in which each individual pellet has layers with different release rates. The drawing shows a core 1.1 with a slow release rate, an outer layer 1.3 with a rapid release rate and an intermediate layer 1.2. Thus, this is a preparation in which a number of different release rates are achieved in each individual pellet.

FIG. 2 shows an embodiment of a capsule according to the invention. Different symbols are used to denote three different groups of active substance pellets which each have different release profiles. Thus the pentagons bearing reference numeral 2.1 represent pellets with a slow release rate, the cubes designated 2.2 are pellets with a moderate release rate and the circles designated 2.3 represent pellets with a rapid release rate. The different active substance pellets of the three groups are delivered to three different absorption sites, where the active substance(s) contained therein are released and absorbed and are able to develop their optimum spectrum of activity.

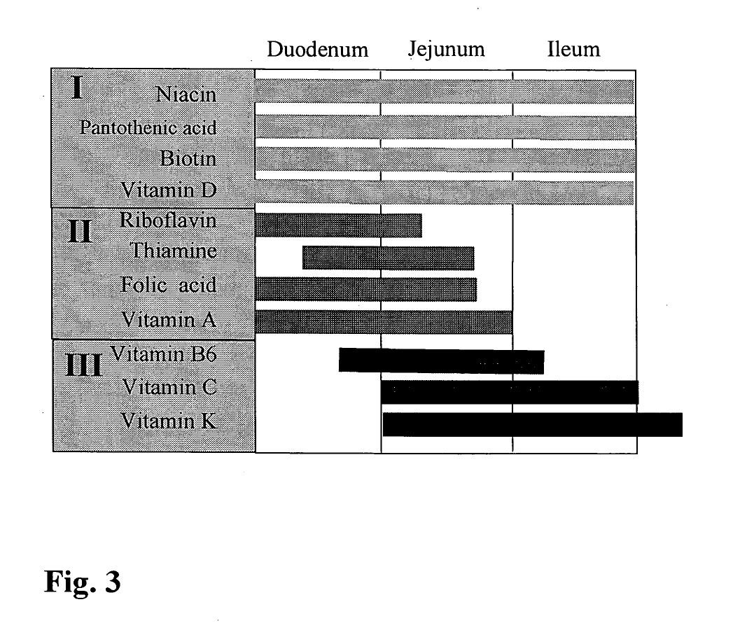

FIG. 3 shows the optimum absorption site or region for selected vitamins in the area of the small intestine. For example, it is found that niacin, pantothenic acid and biotin should be available over the entire region, thus classing them in group I according to the invention, as explained earlier. Accordingly, vitamin C, thiamine, folic acid and vitamin A, which are absorbed in the duodenum and jejunum, may be classed in group II. The vitamins of group III, which are chiefly absorbed in the jejunum and ileum, are vitamin B6, riboflavin, vitamin D, vitamin B 12 and vitamin K shown here. In the case of vitamin B6 it is found that the optimum absorption region is actually in the jejunum and ileum, but may also extend to the duodenum, i.e. also extends to intermediate areas. This may be taken into consideration in the release profiles.

The active substance pellets 1 shown in FIG. 2 may therefore contain one or more vitamins from group I, the active substance pellets 2 may contain one or more vitamins of group II and the active substance pellets 3 may contain one or more vitamins from group III, optionally together with minerals and trace elements as well as other excipients. The active substance pellets 1 shown in FIG. 2 may also contain one or more vitamins from groups I and II and the active substance pellets 2 may also contain one or more vitamins of groups I and III. A number of variants and combinations are possible, particularly if further active substances and nutrients (minerals, amino acids, unsaturated fatty acids) as well as plant extracts and substances are present.

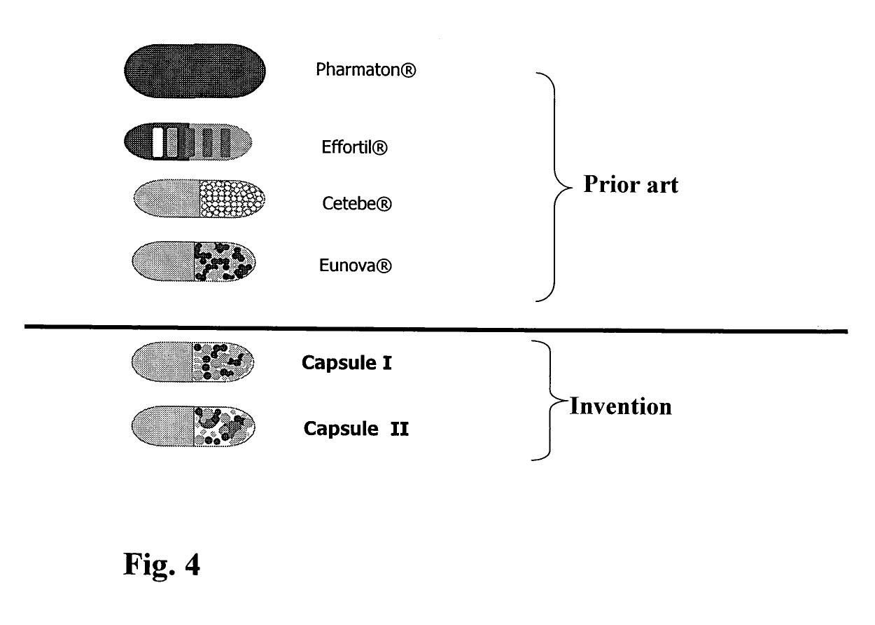

FIG. 4 shows four embodiments known from the prior art, which exhibit a corresponding long-term effect because of the particular formulation used. In the capsule shown at the top, the Pharmaton®Vital capsule, a multivitamin/mineral preparation containing Ginseng, and the second capsule shown, the circulatory drug Effortil® in the form of sustained-release “Perlongette” capsules, both of which may be obtained from Messrs Boehringer Ingelheim GmbH, the long-term effect is achieved by providing a corresponding outer coating on the capsule. In the second and third capsules shown, the vitamin C preparation Cetebe@ and the Eunova@ multivitamin capsule, both of which may be obtained from GlaxoSmithKline Consumer Healthcare GmbH & Co. KG, on the other hand, multi-layer pellets are used, as described for example in WO 01/72286 (cf. also FIG. 1), in order to obtain the sustained or long-term effect.

Moreover, FIG. 4 shows two embodiments of capsules according to the invention which show the different active substance pellets with different release profiles in one capsule. Capsule I shows two groups of active substance pellets and capsule II shows three groups of active substance pellets with different release profiles. The release profiles are therefore time-delayed in the different groups. Thanks to the controlled release targeted on the desired absorption site it is possible to achieve an optimum dosage which leads to optimum absorption of each individual active substance.

The Examples that follow serve to illustrate the formulations according to the invention. They are intended purely as possible procedures described by way of example without limiting the invention to their content.

### EXAMPLES

For the passage through the GIT the following transit times and pH values are assumed in recent literature (Dressman, 2003):

| | pH (on an empty stomach) | transit time |
|---|---|---|
| stomach | 1-3 | 1-2 hours (tablets) |
| | | 30 minutes (pellets) |
| duodenum | 6.0 | 3-4 hours |
| jejunum | 6.5-6.8 | |
| ileum | 7.2-7.5 | |

In the light of these physiological facts and their premise (“Absorption of vitamins in the small intestine”) we propose dividing the ingredients between three types of pellet:

Pellet Type 1: Mineral Pellets

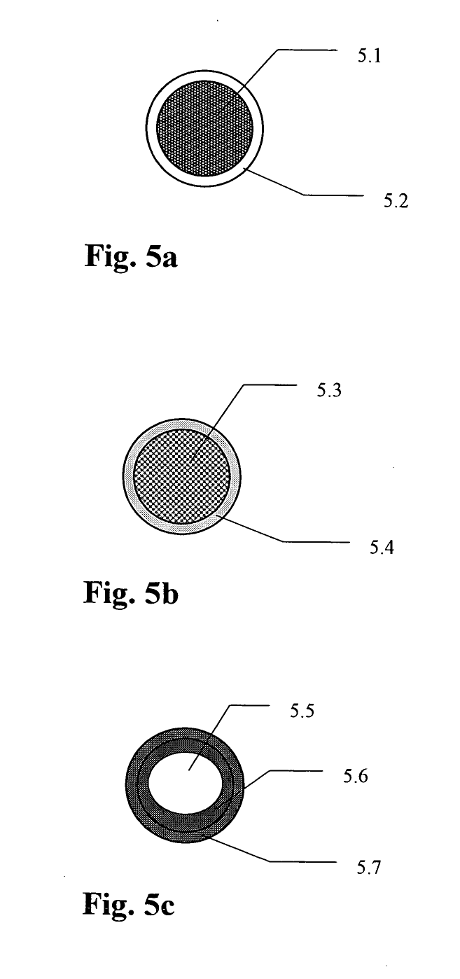

For reasons of stability it is advantageous to separate the minerals from the vitamins. The diagrammatic structure of such a pellet is shown in FIG. 5a.

The mineral-containing pellet core 5.1 is prepared by extrusion and may optionally be provided with a coloured protective film 5.2 of shellac. The coating is not functional. If desired the pellets may also be made resistant to gastric juices by increasing the thickness of the shellac coating.

Pellet Type 2: Vitamin Pellets Group I+II

The diagrammatic structure of this pellet group is shown in FIG. 5b.

The active substance groups (in this case, for example: vitamin groups) I and II (e.g. niacin, vit. A, thiamine, folic acid etc.) should be released over the entire area of the small intestine or preferably as far as the jejunum. This requires a film coating 5.4 which is gastric juice-resistant (GJR), which prevents acid from penetrating into the pellet core 5.3 for 1-2 h and once it has reached the duodenum begins to dissolve and release the vitamins. For example, vitamin groups I and II have the following compositions:

| | | |
|---|---|---|
| group I: | niacin | 16 mg |
| | pantothenic acid | 6 mg |
| | biotin | 50 μg |
| | vitamin D | 5 μg |
| group II: | riboflavin (vit. B2) | 1.2 mg |
| | vitamin A | 800 (μg retinol equivalents) |
| | thiamine | 1.1 mg |
| | folic acid | 400 μg |

The pellet core is also prepared by extrusion and then coated with shellac to make it resistant to gastric juice. The release characteristics are tested in accordance with the corresponding pharmacopoeia monographs.

Pellet Type 3: Vitamin Pellets Group III

The diagrammatic structure of this pellet group is shown in FIG. 5b.

Pellets of type 3 are intended to release their contents at the earliest in the jejunum or later. This requires the use of a delaying membrane 5.6 underneath the GJR coating 5.7, which delays the onset of the release of active substance and also the actual release of active substance. It may be made from substances such as stearic acid, carnauba wax, glycerol behenate or the like. The pellet core 5.5 is also produced by extrusion and may have the following composition, for example:

| | | |
|---|---|---|
| group III: | vitamin B6 | 1.5 mg |
| | vitamin C | 100 mg |
| | vitamin K | 75 μg |

Alternatively it is also possible to produce a matrix with these lipophilic additives or simply to increase the film thickness of the GJR coating.

The release characteristics are again adjusted and tested in accordance with EuAB or USP.

Mixing the Pellets and Filling the Capsules

The three differently coloured types of pellet obtained are mixed together and packed into a size 0 or 0el hard gelatine capsule (or alternatively into 2× size 1).

The precise qualitative and quantitative composition of the vitamins and minerals may be flexibly adapted to the particular needs or requirements.

1. Capsule containing active substance pellets which differ in their release profile in the gastro-intestinal tract, these pellets containing at least two different active substances which are selected from among the vitamins, minerals, trace elements, unsaturated fatty acids, amino acids and/or plant extracts and substances, characterised in that at least three groups of pellets each having the same release profile are contained and the release of the particular active substance takes place over the entire absorption range in the gastro-intestinal tract (group I), in the duodenum or in the duodenum and in the jejunum (group II), or in the jejunum, in the jejunum and ileum or in the ileum (group III).

2. Capsule according to claim 1, characterised in that the different release profiles represent rapid, moderate and/or slow dissolving of the active substance pellets.

3. Capsule according to claim 1 or 2, characterised in that the active substance pellets are coated and the thickness and/or composition of the coating determine the different release profiles.

4. Capsule according to claim 1, 2 or 3, characterised in that each pellet has a selected release profile and the vitamins, minerals, trace elements, unsaturated fatty acids, amino acids and/or plant extracts and substances contained therein are selected in accordance with the release profile.

5. Capsule according to one of the preceding claims, characterised in that the vitamins of group I are selected from niacin, pantothenic acid, biotin and vitamin D.

6. Capsule according to one of the preceding claims, characterised in that the vitamins of group II are selected from vitamin A, thiamine, vitamin B2 and folic acid.

7. Capsule according to one of the preceding claims, characterised in that the vitamins of group III are selected from vitamin B6, vitamin C and vitamin K.

8. Capsule according to one of the preceding claims, characterised in that each active substance pellet contains either vitamins and/or minerals and/or trace elements and/or amino acids and/or unsaturated fatty acids and/or plant extracts/substances of group I, II or III.

9. Capsule according to one of the preceding claims, characterised in that two types of active substance pellets are present, one type comprising vitamins, minerals, trace elements, unsaturated fatty acids, amino acids and/or plant extracts and substances of groups I and II and the other type comprising substances of groups I and III.

10. Capsule according to one of the preceding claims, characterised in that the minerals and trace elements are selected from calcium, magnesium, iron, zinc, copper, potassium, manganese, selenium, chromium, fluoride, phosphorus and iodine, the salts thereof and mixtures thereof.

11. Capsule according to one of the preceding claims, characterised in that the plant extracts are selected from Ginkgo biloba, Panax Ginseng, Vitis vinifera, Guarana, Cimicifuga racemosa, Turnera aphrodisiaca, special plant extracts rich in kaempferol or kaempferol glucosides and/or rich in lutein or other flavonoids.

12. Capsule according to one of the preceding claims, characterised in that the amino acids are selected from lysine, arginine and taurine.

13. Capsule according to one of the preceding claims, characterised in that the unsaturated fatty acids are ω-fatty acids.

14. Process for preparing the capsules according to one of claims 1 to 13, comprising
selecting the active substances on the basis of their respective absorption sites;
producing at least three groups of active substance pellets with different release profiles for the respective absorption sites, incorporating the vitamins and optionally other active substances which are divided between the groups of active substance pellets according to their release profiles and absorption sites, and
introducing the active substance pellets into a capsule.
